(19) United States
(12) Patent Application Publication
(10) Pub. No.: US 2002/0147499 A1
(43) Pub. Date: Oct. 10, 2002

(54) Locking systems for implants

(76) Inventors: Jeffrey J. Shea, Memphis, TN (US); Stephen T. Miller, Germantown, TN (US)

Correspondence Address: CHIEF PATENT COUNSEL, SMITH & NEPHEW, INC., 1450 BROOKS ROAD, MEMPHIS, TN, 38116, US

(21) Appl. No.: 10/083,093

(22) Filed: Feb. 26, 2002

Related U.S. Application Data
(60) Provisional application No. 60/271,517, filed on Feb. 26, 2001.

(51) Int. Cl. A61F 2/32

(52) U.S. Cl. 623/22.21

(57) ABSTRACT

Methods, systems and devices for prosthetic implants having openings that receive insertion members for securing the implants in place. The implants have at least one opening with a tapered section. The insertion members have a head that is spherical, near spherical, toroidal, elliptical, global, or otherwise slightly curved or rounded portion at the outer edge, and are adapted to cooperate with the at least one opening to form a liquid-tight seal.

This application claims priority to Provisional Application No. 60/271,517 filed Feb. 26, 2001 entitled, “Spherical Head Locking Peg and Hole Cover for Acetabular Cup,” which is incorporated herein by reference.

### BACKGROUND OF THE INVENTION

1. Field of the Invention

The present invention relates generally to orthopedic implants, and more specifically, to methods, systems and devices for prosthetic implants having improved locking systems.

2. Description of Related Art

Artificial implants or prostheses, including hip joints, knee joints, and shoulder joints are widely used in orthopedic surgery. Such prostheses are implanted to repair or reconstruct all or part of an articulating skeletal joint that is functioning abnormally due to disease, trauma, or congenital defect. For example, the hip and knee joints are major weight bearing joints that degenerate more quickly than other joints in the event of abnormality.

Hip joint prostheses are common. A hip is actually a ball and socket-type joint, which interfaces two separate bones—the femur and the pelvis, each having a smooth articulating surface. The pelvis has two cup-shaped depressions, called the acetablua or “sockets.” The head of the femur is ball-shaped and the “ball” fits into the “socket,” forming a joint which allows the leg to articulate forward, backward and sideways through a wide, three dimensional, range of motion.

The acetabulum is lined with cartilage, which cushions the bones and allows the joint to rotate smoothly and with minimal friction. An envelope of tough ligaments connects the pelvis and femur, covering the joint and stabilizing it. The cartilage also lends strength to the hip joint in order to support the weight of the upper body, and resilience to absorb the impact of exercise and activity. A healthy hip will allow the leg to articulate freely within its range of motion, while supporting the upper body and absorbing the impact that accompanies activities like running and jumping.

Various degenerative diseases and injuries may require replacement of all or a portion of a hip using synthetic materials. For example, one or both of the articulation surfaces of the hip joint may fail to perform properly, requiring the defective natural articulation surface to be replaced with a prosthetic articulation surface provided by an implantable prosthesis. A range of orthopedic implants is available to accommodate various defects, while permitting healthy portions of the hip joint to be conserved. For example, there are total hip prosthesis systems for replacing the articulation surfaces of both the femur and the pelvis; less comprehensive systems for replacing only the femoral articulation surface are also provided.

Total hip arthroplasty and hemi-arthroplasty are two procedures well known within the medical industry for replacing all or part of a patient's hip. A total hip arthroplasty replaces both the femoral component and the acetabular surface of the joint, so that both a femoral prosthesis and an acetabular prosthesis are required. A conventional acetabular prosthesis may include a cup, a cup and a liner, or in some cases only a liner, all of which may be formed in various shapes and sizes. Hemi-arthroplasty refers to replacing part of a hip joint, such as replacing a femoral component so that a femoral prosthesis articulates against natural body tissue in the patient's acetabulum.

Various cups, liners, shells, stems and other components may be provided in each type arthroplasty to form modular prostheses to restore function of the hip joint. During surgery, implant components especially selected to match the patient's needs are affixed to the area between the femur and the pelvis. In most cases, the implant will consist of two pieces: a femoral prosthesis metal stem fitted with a ball at one end (“the head”) and a metal or polyethylene cup (“the cup”). There may also be a liner associated with the cup.

More specifically, femoral prostheses used in total hip arthroplasty generally include a spherical head attached to an elongate stem with a neck connecting the head and stem. In use, the elongate stem is located in the intramedullary canal of the femur and the spherical head articulates relative to the acetabular component. Femoral prostheses used in total hip arthroplasty procedures may or may not differ from an endoprosthesis used in a hemi-arthroplasty, described below.

However, the femoral head of each type prosthesis is generally a standard size and shape. The head may be formed of metallic material, polymeric, ceramic or other desired material. It fits into an acetabular cup which has been inserted in the acetabulum of the patient. The cup may include a liner such as a polymeric liner to receive the head. The liner and cup components may articulate or not relative to each other; the head articulates relative to the liner. These components replace the ball and socket of the femur to form a new hip joint.

Consider the acetabular cup and liner. The cup is usually of generally hemispherical shape and features an outer, convex surface and an inner, concave surface that is adapted to receive a cup liner, which is typically polymeric, ceramic, or metal. The liner fits inside the cup and has a convex and concave surface. The cup liner is the bearing element in the acetabular component assembly. The convex surface of the liner corresponds to the inner concave surface of the cup or acetabulum, and the liner concave surface receives the head of a femoral component. An acetabular cup may include a highly polished inner surface in order to decrease wear.

During surgery, an acetabular prosthesis may be fixed in the reamed acetabulum of a patient. The pelvis is prepared to receive the acetabular cup by reaming a concavity in the acetabular bone. As described, the acetabular cup component typically has an outer surface conforming to the concavity reamed in the acetabular bone of the pelvis, and an inner bearing cavity for receiving the head of the femoral component. The head articulates in the bearing cavity as a ball-and-socket joint to restore motion to a defective hip joint.

Some acetabular cup prosthetic devices are also secured into the acetabular bone by projections on the outer surface of the cup which are a part of the prosthesis as constructed. Pegs which are pre-attached to the cup may require pre-drilling or other preparation which includes impaction for the purpose of forcing the projections into the bone. The bone bed may also require advance preparation in some devices to accept the protrusions. Additionally or alternatively, bone cement may also be used alone or in conjunction with such projections or pegs in order to more fully secure the cup in place.

Alternatively, the cup may be affixed to the acetabular bone by bone screws or pegs that are placed through the cup. Generally, the surgeon will select a cup having a series of holes or openings depending on how serious the bone deficiency is. It may only be necessary to use one screw or one bone peg to secure the cup in place or in some cases, it may be necessary to use a plurality of screws, modular pegs, or spikes.

Accordingly, acetabular cups are generally provided with either corresponding screw holes, peg holes, or spike holes. These holes are typically designed for use specifically with either a peg, screw, or spike for affixing the cup to the patient's acetabulum, but screws, pegs and spikes are not intended to be interchangeable. In other words, holes designed for use with a screw do not accommodate a peg, and similarly, holes designed for use with a peg do not adequately accommodate a screw.

Some surgeons implant pegs in screw holes and/or screws in peg holes with less than optimal results. For example, FIG. 1A shows an example of a “V-shaped” hole of the prior art. FIG. 1B shows that the use of “V-shaped” hole 10 requires precise alignment between the hole and peg 12. FIG. 1C shows that although “V-shaped” hole 10 is not particularly configured to receive a screw, use of screw 24 in “V-shaped” hole 10 also requires precise alignment, and the screw is not easily inserted at an angle.

The are also provided holes that are particularly designed to receive screws. FIG. 2A shows a “for screw only” (“FSO”) hole 30 which has a circular or curved portion 32 that extends the substantial portion of the inner surface of the hole 30. Although the curved surface 32 allows a screw 24 to be implanted at an angle relative to the central axis 34 of the hole 30 and maintain the head within the FSO hole 30, this type of hole does not lock the screw 24 in place, nor is it adapted to receive a peg 12.

Moreover, because there are commonly provided more screw holes and peg holes than the surgeon will use, some of the holes may receive a screw or peg while others do not. Cups having a number of holes provide a selection of sites for placement of the bone screws, as may be dictated by the condition of the patient's pelvic bone or by the physician's preference. After the bone screws or pegs are driven into the bone through the holes in the cup before the liner is placed into the cup, it is desirable to provide a cover for the holes that are not in use to prevent migration of wear debris from the cup to the patient's bone. Covering the holes also prevents migration of cement if used to secure the cup into the acetabulum.

Generation of wear debris can be a major problem in prosthetic implants. For example, although liners often include locking tabs or other means for fixing the liner into the cup in nonarticulating relative relationship, a small amount of unintended relative motion is believed to occur between the liner and the cup in response to the varying load borne by the acetabular cup during use. Such small relative motion, or micro-motion, may result in wear at the interface between the bearing insert and acetabular shell that generates fine polyethylene or metal debris. This type of wear causes the need for increased replacements and revisions, which are commonly more labor intensive, more expensive, and more traumatic to the surrounding tissue and bone.

Wear debris has been associated with adverse biological responses which can lead to local cell death (osteolysis for bone cells), premature loosening and failure of orthopaedic devices, and subsequent need for revision surgery. The majority of wear debris originates from articulating surfaces of orthopaedic devices, typically an ultra high molecular weight polyethylene insert or liner that is disposed against a metal or ceramic plate or ball surface so that the surfaces engage in articulating motion relative to each other. Additionally, abrasive third body debris, such as bone cement (for example, polymethylmethacrylate bone cement) and bone debris may migrate to the interface between bearing or articulating surfaces, further accelerating abrasive wear due to motion. Debris generated from this wear may migrate out of the acetabular cup and contact bone, possibly resulting in osteolysis, which ultimately can lead to bone resorption and possible loosening of the acetabular prosthesis.

Debris may also be generated by contact between the retaining bone screws or pegs and the liner, particularly if the surgeon has placed a screw in a hole that is shaped to receive a peg. For example, consider an acetabular cup with a bone screw hole with a typical bore or hole and a typical corresponding screw or peg having a tapered section. If the bone screw is not placed precisely so that the head of the screw cooperates with the shoulders of the bore, for example, if the surgeon does not precisely drill or if the screw is placed at an angle, as the patient walks on the new hip replacement, the cup may settle into the bone while the screw stays in place, causing the head of the screw to slowly start wearing against the polyethylene liner, generating wear particles. One pathway for the migration of debris out of the acetabular shell is through open apertures that have not been sealed by a screw, peg or spike. Another pathway for migration of the debris is through a failed or loose connection between a bone screw, bone peg, bone spike, or hole cover because the structure used did not correspond appropriately with the hole receiving the structure. Even with properly fitting screws, some wear debris may be allowed to migrate through the screw hole and become deposited between the cup and the acetabulum. Accordingly, it is desirable that these connections be liquid-tight.

There is not currently provided a “universal-type” locking design that is interchangeable, in that it can accommodate any one of a bone screw, a bone peg, a bone spike, or a hole cover in order to provide the surgeon with the greatest flexibility. There is also not currently provided an optimal hole configuration that allows a securing mechanism to be inserted at an angle, yet maintain a secure connection to prevent migration of debris, while also maintaining the head of the screw within the hole.

Therefore, it is desirable to provide implants, and particularly acetabular cups, having “universal-type” holes or openings that can receive and lock any one of a peg, screw, spike or hole cover in place. This would provide surgeons with option of using either a screw, peg, or spike to secure the cup in place, depending upon the patient's anatomy, without having to choose an alternate cup or choose an alternate securing device.

Another common joint replacement surgery is total or partial knee replacement. The human knee joint involves three bones: the femur, the tibia and the patella, each having smooth articulation surfaces for articulation on or against an adjacent articulation surface of at least one other bone. All or part of one or more of the articulation surfaces of the knee joint may fail to perform properly, requiring the defective natural articulation surface to be replaced with a prosthetic articulation surface provided by an implantable prosthesis. A range of orthopedic knee implants is available to accommodate defects of varying scope, while permitting healthy portions of the knee joint to be conserved. The range extends from total knee prosthesis systems for replacing the entire articulation surface of each of the femur, tibia and patella, to less comprehensive systems for replacing only a portion of the joint.

In knee joint replacement surgery, a surgeon typically affixes prosthetic components to the patient's bone structure; a first component to the patient's femur and a second component to the patient's tibia. These components are typically known as the femoral component and the tibial component, respectively. Each component may be formed of a range of subcomponents, such as in a modular fashion. For instance, a tibial tray that corresponds in some ways to the tibial plateau may be supported in some prosthetic designs by a cemented or non-cemented tibial stem that is inserted into the canal of the tibia. Similarly, the condylar component can be supported by a stem or other structure that attaches to or is inserted into the femur.

The femoral component is placed on a patient's distal femur after appropriate resection. The femoral component is usually metallic, having a highly polished outer condylar articulating surface. A common type of tibial component uses a tray or plate that generally conforms to the patient's resected proximal tibia. The tibial component also usually includes a stem which extends generally perpendicular to the plate in order to extend into a surgically formed opening in the patient's intramedullary canal.

A healthy knee joint flexes, extends, and rotates as a person walks, sits, bends forward, and climbs stairs. Nature has provided a self-lubricating system of healthy, white cartilage to cover the ends of these bones. It is this smooth, slippery surface that enables the knee to glide like a well-oiled machine with no rough spots to interfere with its precise, rhythmic motion. In the implant context, this smooth, slippery surface is removed and replaced with a plastic or polymeric (often high density polyethylene or ultra high molecular weight polyethylene) insert or bearing. The insert fits between the tibial component and the femoral component and provides a surface against which the femoral component condylar portion articulates.

Tibial plates of the type described can be affixed to the resected tibial bone by bone screws, bone spikes, bone cement, or a combination thereof. If bone screws or spikes are elected, they are driven into the bone through screw or spike holes in the plate before the bearing insert is placed atop the plate portion. The plate also can be affixed by a combination of bone screws, spikes and/or bone cement. It is conceivable that bone pegs may also be used in some embodiments.

The same wear problems described above with respect to hip joints can be experienced with a knee joint replacement. For example, if screws are placed into a hole not specifically adapted to receive a screw, there is a high possibility that the screw may wear against the bearing insert. Additionally or alternatively, if the screw is placed at an angle, it may protrude above the surface of the tibial plate. Moreover, if a surgeon desires to use a peg in a tibial plate that can only accommodate screws, the surgeon is unduly limited in his choices of treatment.

As with acetabular cups, the plate may also be provided with more screw holes than typically would be used by the implanting physician to provide a selection of sites for placement of the bone screws, as may be dictated by the condition of the patient's tibial bone or by the physician's preference. Some of the provided screw holes may receive a screw while others do not. For reasons similar to those discussed above with regard to acetabular cups, it is desirable to provide a universal locking design that can accommodate any one of a bone screw, a bone peg, a bone spike, or a hole cover in order to provide the surgeon with the greatest flexibility.

Similar principles may apply in any number of contexts in any combination of prosthetic implant bearing surfaces, to which the present invention is potentially applicable. For example, although not described in detail, the concepts embodied by this invention are intended for use with any implants, such as shoulder implants, elbow implants, spinal implants, and so forth. More broadly, the present invention is potentially applicable to any device that is adapted to receive a medical screw, such as hip screws, interlocking nails, spinal plates and screws, and the like.

### SUMMARY OF THE INVENTION

Set forth below is a brief summary of systems, methods, and devices according to various embodiments of the invention that address the foregoing problems and provide benefits and advantages in accordance with the purposes of the present invention as embodied and broadly described herein. Generally, the implants or other medical devices provided have at least one opening with a tapered section that preferably comprises a frustoconical taper. Generally, the insertion members provided have a head that is spherical, near spherical, toroidal, elliptical, global, or otherwise having a slight curve or rounded portion at its outer edge; the insertion members are adapted to cooperate with the at least one opening to form a liquid-tight seal.

According to various embodiments of the invention, there is provided an implantable prosthesis, comprising a prosthetic component having first and second surfaces, the second surface adapted to be oriented toward bone in which component is to be implanted; at least one opening extending from the first surface to the second surface; the opening adapted to receive an insertion member and comprising (i) an extended frustoconical taper section extending from the first surface through a substantial portion of the opening and (ii) a section at the second surface having a smaller diameter than the portion of the taper at the first surface, the opening adapted to accommodate an insertion member at multiple orientations relative to the component; and an insertion member having a spherical or near-spherical head and adapted to be inserted into the opening such that the spherical or near-spherical head interfaces with the extended frustoconical taper section.

In a second aspect of the invention, there is provided, an implantable prosthesis, comprising a prosthetic component having first and second surfaces, the second surface adapted to be oriented toward bone in which component is to be implanted; at least one opening extending from the first surface to the second surface; the opening adapted to receive an insertion member and comprising (i) an extended frustoconical taper section extending from the first surface through a substantial portion of the opening and (ii) a rounded section at the second surface having a smaller diameter than the portion of the taper at the first surface, the opening adapted to accommodate an insertion member at multiple orientations relative to the component.

A further aspect of the invention provides an acetabular implant for fixation to a patient, comprising an acetabular cup having an inner surface, an outer surface, and at least one opening extending from the inner surface to the outer surface, the outer surface adapted to be oriented toward bone in which component is to be implanted; the at least one opening having an extended frustoconical tapered section beginning at the inner surface; and a member for insertion into the opening, the member comprising a head having a spherically or near-spherically shaped portion that is adapted to interface with the extended frustoconical tapered section to lock and retain the insertion member at a desired orientation relative to the opening.

There are also provided insertion members for use with a prosthesis having a universal connecting portion, comprising a head having an outer edge shaped to approximate at least a portion of a sphere, a near-sphere, a toroid, an ellipse, a globe, a slight curve, or rounded portion ; and a portion extending from the head adapted to be received by an opening of the prosthesis.

Another aspect of the invention provides an acetabular implant for fixation to a patient, comprising an acetabular cup having an inner surface, an outer surface, and at least one opening extending from the inner surface to the outer surface, the at least one opening having an extended frustoconical tapered section beginning at the inner surface and a second section at the outer surface having a diameter smaller than the diameter at the inner surface; a member for insertion into the opening, the member comprising a head having a spherically or near-spherically shaped portion that is adapted to interface with the frustoconical extended tapered section to lock and retain the insertion member at a desired orientation relative to the opening.

Also according to various embodiments of the invention, there is provided a method of replacing at least part of a hip joint in a patient, comprising:

(a) providing an acetabular cup having an inner surface, an outer surface, and at least one opening extending from the inner surface to the outer surface; the opening adapted to receive an insertion member and comprising (i) an extended frustoconical taper section extending from the first surface through a substantial portion of the opening and (ii) a section at the second surface having a smaller diameter than the portion of the taper at the first surface, the opening adapted to accommodate an insertion member at multiple orientations relative to the component; and

(b) providing at least one insertion member having a spherical or near-spherical head and adapted to be inserted into the opening such that the spherical or near-spherical head interfaces with the extended frustoconical taper section;

(c) preparing the bone of the patient's hip to receive the acetabular cup; and

(d) implanting the acetabular cup,

wherein once the appropriate orientation of the insertion member is selected, the insertion member is adapted to be locked relative to the frustoconical taper section.

### BRIEF DESCRIPTION OF THE DRAWINGS

The accompanying drawings, which are incorporated in and form a part of the specification, illustrate preferred embodiments of the present invention and, together with the description, disclose various embodiments of the invention.

FIG. 1A shows a side sectional view of a V-shaped hole of the prior art.

FIG. 1B shows a side sectional view of a V-shaped hole of FIG. 1A with a corresponding prior art peg placed therein.

FIG. 1C shows a side sectional view of a V-shaped hole of FIG. 1A with a prior art screw inserted at a slight angle.

FIG. 2A shows a side sectional view of a “for screw only” opening of the prior art.

FIG. 2B shows a side sectional view of the “for screw only” opening of FIG. 2A with a traditional tapered screw placed therein.

FIG. 3 shows an exploded perspective view of an acetabular cup receiving an insertion member and a liner according to one embodiment of the present invention.

FIG. 4 shows a side sectional view of an opening according to a preferred embodiment of the invention.

FIG. 5 shows a side sectional view of an opening according to an embodiment of the invention with a non-locking bone screw.

FIG. 6 shows a side sectional view of an opening according to an embodiment of the invention with a locking bone screw.

FIG. 7 shows a side sectional view of an opening according to an embodiment of the invention with a locking peg in place in a patient.

FIG. 8 shows a side sectional view of an opening according to an embodiment of the invention with a locking opening cover.

FIG. 9 shows a side plan view of a “universal-type” head of an insertion member according to one embodiment of the invention.

FIG. 10 shows a perspective view of a portion of an insertion member prior to being inserted into a prosthesis.

FIG. 11 shows a perspective view of the insertion member of FIG. 10 inserted into the opening of an prosthesis.

### DETAILED DESCRIPTION OF THE DRAWINGS

Methods, systems and devices according to this invention seek to provide a universal hole or opening geometry that can receive and securely retain in place an insertion member, such as any one of a bone screw, a bone peg, a bone spike, or a hole cover. Methods, systems and devices according to this invention also seek to provide a locking system on insertion members that allows the insertion members to cooperate with such openings in an optimal manner. Various embodiments of this invention are applicable to any type of prosthesis, such as hip prostheses, knee prostheses, shoulder prostheses, and so forth. Embodiments of this invention are also other devices that are adapted to receive a medical screw or other attachment member, such as hip screws, interlocking nails, spinal plates and screws, and the like. For the sake of demonstration, the specific embodiments of the invention described here will focus on hip prostheses, but it should be understood that the descriptions are equally applicable to other prostheses as well.

A plurality of insertion members can be interchangeably and rigidly attached to a prosthesis surface for the purpose of securing the prosthesis in place in or on bone. Specifically, as shown in the drawings, a plurality of insertion members can be interchangeably and rigidly attached to an acetabular cup prosthesis body for the purpose of securing the cup in place in the acetabular bone. As used herein, the term “insertion member” includes but is not limited to bone screws, bone pegs, bone spikes, opening covers, and any other member intended to be at least partially inserted into an opening of a prosthesis. This is done through an opening which can be interchangeably used for a desired bone screw, bone peg or bone spike. Unused openings can be covered with an opening cover that includes the same securing mechanism at its head as that of the screws, pegs, or spikes.

During surgery, an acetabular cup can be placed in the desired position in the acetabulum by the surgeon. Insertion members are then inserted through openings of the cup and attached to the prosthesis in a rigid fashion. Each insertion member protrudes through the acetabular cup body and into the underlying bone tissue of the acetabulum to provide a mechanical locking of the acetabular cup into the pelvis. Alternatively, the insertion members are opening covers that cover unused openings. In this instance, the insertion member does not necessarily protrude through the acetabular cup, but instead lies substantially flush with the surface of the cup that faces bone. The surgeon can pre-drill the tissue before placing the insertion member into the cup and the openings in the acetabular cup body typically function as a drill guide.

In some instances, pegs, screws or spikes can be selectively placed so that they are not aligned with each other, but are at angles to each other, which aids in the mechanical stability of the acetabular cup body. The problems encountered in this situation are many. Non-limiting examples of such problems include (a) the head of the pegs, screws, or spikes may extend above the opening and wear against the liner; (b) the desired liquid-tight interface between the opening and the peg, screw, or spike may be compromised because the angle causes a slight opening at the locking interface; and (c) traditional openings are provided for either pegs, screws, or spikes and a surgeon may wish to use an alternate insertion member. Accordingly, the present invention provides preferred configurations that seek to alleviate at least some or all of these problems.

The preferred embodiment of the invention preferably comprises a plurality of insertion members that feature a head that is adapted to interface with an opening having an extended frustoconical taper. In a specific embodiment, the head of the insertion member is spherical, near spherical, toroidal, elliptical, global, or otherwise has a slight curve or rounded portion at its outer edge. For the purposes of this document, such head configurations will collectively be referred to as spherical or near-spherical. It should be understood, however, that a perfect sphere shape, toroidal shape and so forth is not required in order to practice this invention. Slight manufacturing tolerances may prevent the formation of a perfect sphere. Moreover, fluctuations in the shape of the spherical or near-spherical head will not prevent the locking function desired to be achieved by the present invention.

The interface between the insertion member and the frustoconical taper of the opening forms an interference fit, or compression friction lock, to provide a rigid connection with the acetabular cup at the tapered surface. The interference fit assures a rigid connection between the insertion member and the cup body so that the insertion member and the cup body move together, if at all, rather than relative to one another. The interference fit assures a rigid connection when the insertion member is placed at an angle, due to the geometry between the taper and the head that is provided. The illustrated acetabular cups have locking systems that are particularly advantageous because they provide a “universal-type” opening that cooperates with corresponding insertion members having a “universal-type” head configuration.

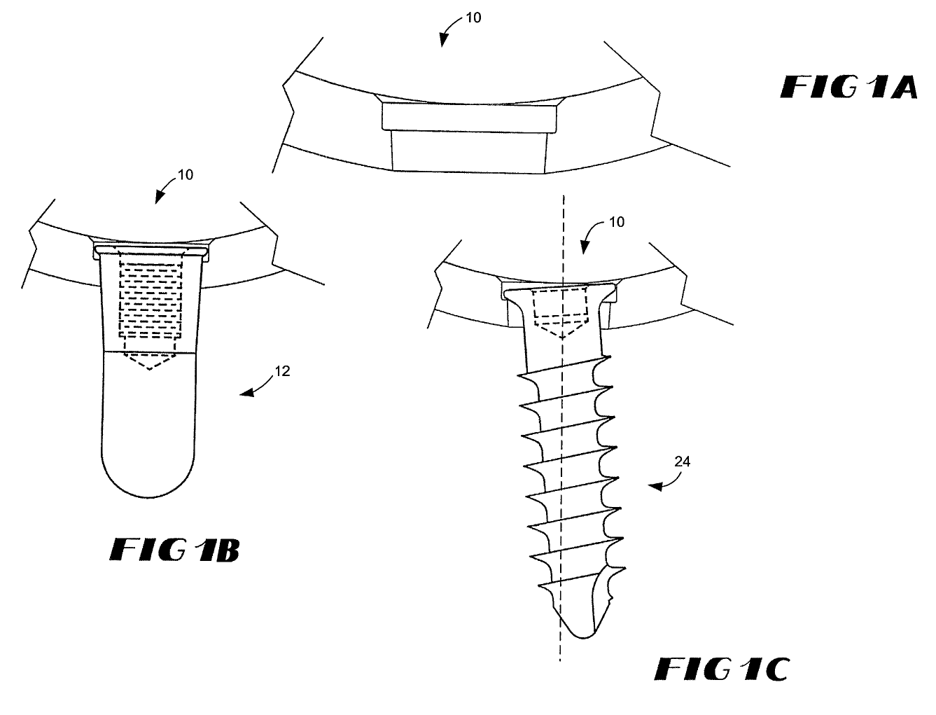

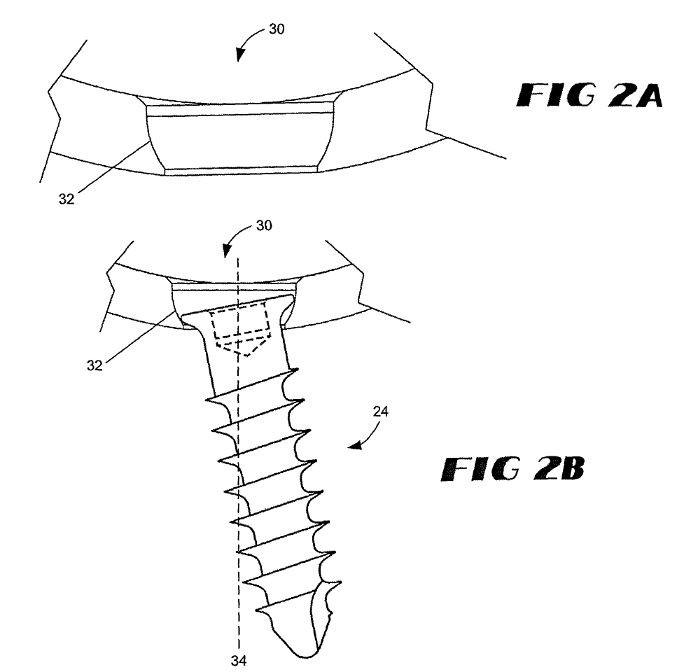

Referring now to the drawings, FIGS. 1 and 2 show generally the prior art hole configurations described above. Generally, these hole geometries limit surgeons because the holes are specifically shaped to receive either a peg, a screw, or a hole cover having the head configuration of either a peg or a screw. If a screw is placed in a peg hole, there is a risk that the screw may not be placed properly and it is unlikely that a liquid-tight seal will result, leading to the possibility of migration wear debris. Moreover, the screws, pegs and hole covers of the prior art are not provided with heads having a “universal-type” geometry, that is preferably a spherical or near-spherical shape, that facilitates the enhanced lock, even at angles, that is provided by the present inventors.

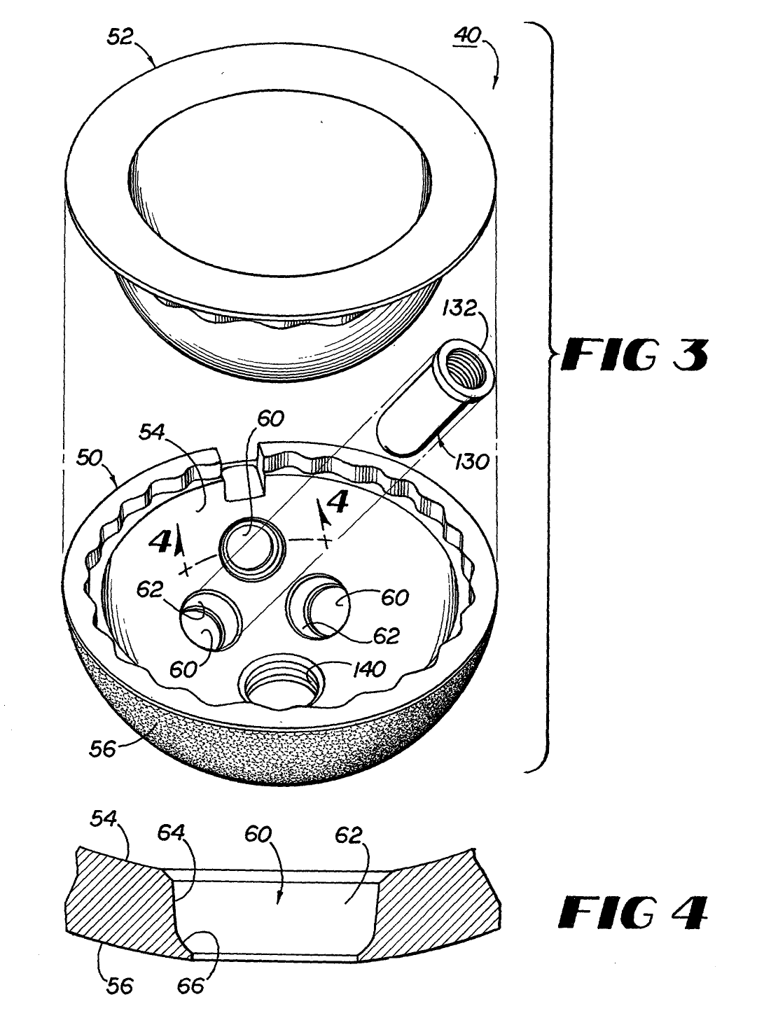

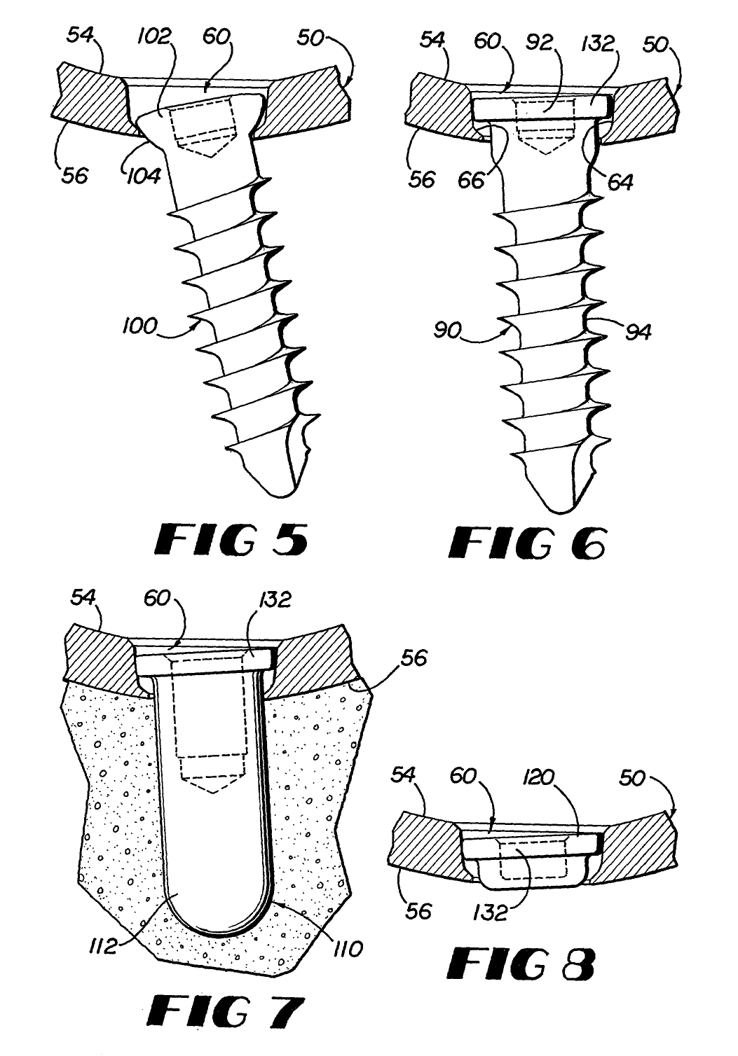

Accordingly, FIGS. 3-9 show an improved locking system. It is worth noting again that although the Figures and Detailed Description of the Drawings relate to hip prostheses, the locking system of this invention is equally applicable to other joint prostheses. FIG. 3 shows generally an implantable orthopedic prosthesis assembly 40 according to one embodiment of the present invention. Particularly, FIG. 3 shows an acetabular cup 50 component and bearing liner 52 of a total hip joint prosthesis.

Acetabular cup 50 preferably comprises a cup of a biocompatible metallic material, such as titanium, titanium alloy, stainless steel, cobalt-chrome alloy, or combinations thereof. However, any metal material that is sufficiently strong and sufficiently biocompatible may be used and it is understood that various additional materials may meet these parameters. Cup 50 may be spherical in form as shown, or not spherical in form (such as e.g., an egg-shaped cup).

Cup 50 has a first surface 54 that is a smooth, inner concave surface adapted to receive a cup liner 52. The cup liner 52 is in turn adapted to receive a femoral stem (not shown). Only the acetabular cup 50 will be described in detail, as the various types and configurations of cup liners and femoral stems for use in conjunction with the cup are well understood in the art and may be used with the present invention. Additionally, the fixation of such bearing liners within an acetabular cup is well understood in the art and will not be described further. An exemplary fixation method can be found in U.S. Pat. No. 5,310,408, incorporated herein by reference.

First surface 54 is preferably a highly polished inner surface in order to decrease wear that may be generated due to micro-movement of the liner 52. See e.g., U.S. Pat. No. 5,310,408. However, the highly polished inner surface, while preferred, is not necessary for the practice of the invention.

Cup 50 also has a second, outer convex surface 56 that is preferably textured to promote bone ingrowth. For example, second surface 56 is shown having a roughened or porous coated surface. This helps promote bone ingrowth once cup 50 has been positioned in a patient for a more secure fit. Surface 56 may comprise sintered beads, plasma sprayed metal, plasma sprayed hydroxyl apatite, or a mechanically roughened or textured surface. Again, while the textured or porous coating is preferred, it is not necessary for the practice of the invention.

Acetabular cup 50 is also typically provided with a dome hole 140 at the apex of the cup 50. Often, the dome hole 140 is internally threaded or otherwise configured for receiving an instrument for holding and positioning the acetabular cup 50 during implantation. Also, many surgeons use the dome hole 50 to obtain visual or tactile access to the reamed acetabular bone during implantation of the acetabular cup. Such access allows the physician to confirm that the acetabular cup is fully seated in engagement with the reamed bony surface of the acetabulum.

Dome hole 140 typically receives a hole cover, but may also receive a bone screw or other fixation device to secure cup 50 in place. Moreover, dome hole 140 is usually a differently shaped hole from openings 60 (as shown) but it is understood that dome hole 140 may also be shaped the same as openings 60, further providing more flexibility to the surgeon and enabling the use of the “universal-type” insertion members described.

Cup 50 also includes a plurality of holes or openings 60 extending between the first inner surface 54 and the second outer surface 56. Although three such openings 60 are shown, it is understood that more or less openings may be provided. The plurality of openings 60 may act as drill guides so that during surgical implantation of the prosthesis, the surgeon can selectively drill into the underlying tissue through one or more of openings 60 and form surgical openings in the underlying bone tissue. Openings 60 also receive insertion members that either secure cup 50 into the patient's bone or cover un-used openings.

Openings 60 preferably have a similar shape and geometry and are typically positioned in one quadrant of cup 50. Each opening 60 is adapted to receive any one of the insertion members described herein, such any one of a bone screw, a bone peg, a bone spike, or an opening cover. In general, openings 60 have inner walls 62 with an extended frustoconical taper section 64.

Taper section 64 is preferably a self-locking taper section. In a specific embodiment, taper section 64 has a taper angle that is typically 20° or less. In an even more specific embodiment, taper section 64 has a taper angle that is 10° or less. The angle of the slant is not critical to the invention, however, it is preferred that the taper angle be sufficient that a head of an insertion member can be received and locked therein, thus providing at least a partial seal.

As shown in FIG. 4, non-threaded taper section 64 extends from first surface 54 through a substantial portion of opening 60. This extended taper section 64 provides a locking area for receiving and securing heads of insertion members according to various embodiments of this invention. It may also be possible to use openings 60 of this invention for securing the prior art screws and pegs discussed above, although the geometry of the insertion members described herein provides a more secure and liquid-tight lock.

Openings 60 also have a smaller diameter section 66 at or near second surface 56. Although smaller diameter section 66 is shown as a curved edge, this shape is not critical to the invention. Section 66 may be any shape, such as a flat edge, a chamfered edge, a beveled surface, or as shown, a rounded or curved surface. It is merely preferred that smaller diameter section 66 have a smaller diameter than the diameter of taper section 64 at first surface 56. In particular embodiments, smaller diameter section 66 is curved or rounded to facilitate positioning of a non-locking screw or other member. In even more particular embodiments, if section 66 is rounded, it may be provided with a spherical or non-spherical surface.

For example, if smaller diameter section 66 of opening 60 is rounded, it is more particularly shaped to receive at least a portion of corresponding rounded portion 104 of a non-locking head screw 100, as shown in FIG. 5. In this embodiment, portion 104 and opening 66 may be curved, spherical, or any other shape that provides at least some sort of rounded portion that can interface with another rounded portion. Because non-locking bone screws 100 are specifically designed to be inserted at an angle and thus, should be rotatable in opening 60, non-locking screws 100 have head 102 with a substantial rounded portion 104 that allows non-locking screw 100 to rotate substantially in opening 60. Thus, non-locking screws 100 may require a greater amount of space in opening 60 in which to maneuver prior to securement to ensure that the head 102 does not extend past first surface 54 and impinge on the liner in use. By providing extended tapered section 64 proximally (closer to the first surface 54) and the smaller diameter section 66 as a rounded surface and located distally (closer to second surface 56), a non-locking head bone screw 100 may be accommodated in an optimal manner. The head 102 of screw 100 remains fully within the recess of opening 60 when the screw 100 is inserted at an angle. This allows the surgeon to place the screw 100 at a proper angle, without compromising the position of the screw 100 with respect to the cup 50 or its placement within the acetabulum. In use, rounded portion 104 interfaces with smaller diameter section 66. This interface may be accommodated even if smaller diameter section 66 is a geometry other than rounded, but a rounded section is preferred for most non-locking embodiments.

Openings 60 are also configured to also receive insertion members according to various embodiments of this invention. Specially adapted insertion members, such as sealing or locking screws, pegs, spikes, or opening covers, having a proprietary head configuration provided by this invention and described in detail below. Briefly, as shown in FIG. 6, extended taper section 64 provides a recess that can receive head 92 of screw 90 when screw 90 is inserted through opening 60 at an angle, and still provide sufficient clearance between head 92 and first surface 54 such that locking of screw 90 at an angle will not cause screw head 92 to project past first surface 54 and into the concave interior of cup 50, causing generation of wear particles.

In some embodiments, however, it is not necessary that head 92 of screw 90 be retained entirely within opening 60. One non-limiting example is if the 50 is used without a liner, because there is no concern about the generation of wear from the head 92 against the liner. Accordingly, devices in which head 92 protrudes past first surface 54 are also considered within the scope of the invention.

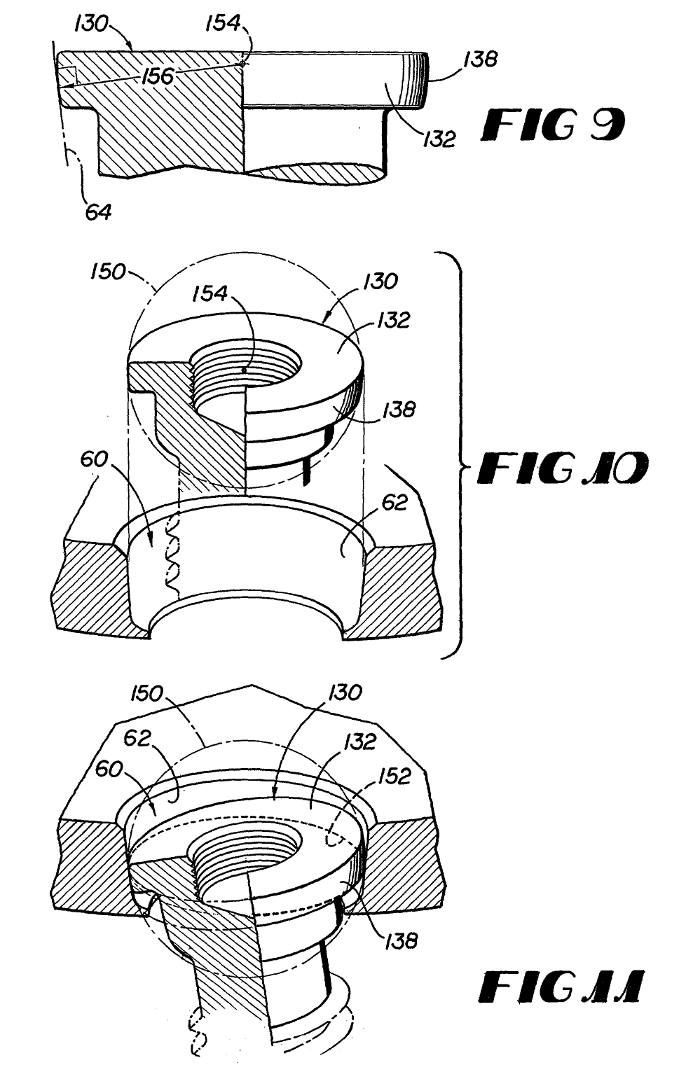

Moreover, surgeons may wish to use various insertion members depending upon many factors, such as the severity of a patient's condition, the strength of the underlying bone, the surface area available and so forth. For example, bone pegs 110, such as that shown in FIG. 7, are smooth along the extended portion 112 so that movement of the peg 110 and cup 50 as a unit will not disrupt adjacent bone tissue. On the other hand, bone screws 90, such as that shown in FIG. 6, have a threaded portion 94 that allows screw 90 to be securely implanted into bone. (Note that additional securing or insertion mechanisms other than those shown in the Figures may be used and are considered within the scope of this invention. For example, bone spikes or various other configurations for other securing members may be used. For example, bone pegs having a porous coating, screws having any number or shape of threads, and so forth, are considered within the scope of this invention.) In use, insertion members are insertable into and register with openings 60 in the cup 50. As detailed in FIGS. 9-11, the basic head configuration of insertion members is common for all types of insertion members according to the various embodiment of this invention. Accordingly, for the ease of description, a general insertion member 130 is shown and described.

Insertion member 130 generally has a spherical or near-spherical head portion 132. It should be understood that the terms “spherical” and “near-spherical” are used to refer to head portions 132 that have an outer rim portion 138 that is spherical, near spherical, toroidal, elliptical, global, or that otherwise has a slight curve or rounded portion at its outer edge. Head portion 132 is not required to be perfectly spherical, toroidal, elliptical, or global in order to practice this invention. Specifically, it is understood that slight manufacturing tolerances may prevent the formation of a perfect sphere or other circular geometric shape. Fluctuations in the shape of the spherical or near-spherical head will not prevent the locking function desired to be achieved by the present invention.

In use, head portion 132 forms a rigid connection with a prosthesis, such as acetabular cup 50, at one of the openings 60. Once insertion member 130 has been placed into opening 60, head portion 132 abuts frustoconical taper 64 of inner wall 62. In other words, the extended taper portion 64 of inner wall 62 cooperates with the spherical or near-spherical shape of head portion 132 to secure insertion member 130 in opening 60. The cooperation is that of a frustoconical taper/at least partial sphere interface.

More specifically, in a particular embodiment of the invention, head portion 130 is designed so that insertion member 130 fits into opening 60 and can be optimally angled while maintaining a secure connection at the interface between inner wall 62 and head portion 132. For example, refer to FIG. 10 and consider a sphere, such as a round ball. If the sphere 150 was placed into opening 60, there would be an entire circle of contact around sphere 150 where it interfaces with inner wall 62. No matter how sphere 150 is rotated or angled, the circle of contact would always remains in contact with inner wall 62. The phantom sphere 150 inserted into opening 60 and the resulting circle of contact 152 (shown in phantom) is shown in FIG. 11.

Although head portion 132 may be a complete sphere or a near-sphere, in some instance, for obvious reasons, it is not workable to use an entire sphere for spherical head 132 of insertion member 130. For example, if a liner is being used, the top of the sphere 150 would interfere with the insertion of liner 52 and the bottom of sphere 150 would interfere with the placement of the prosthesis against the patient's bone. If a liner is not being used, it may be desirable to retain the top portion of sphere 150, but remove the bottom portion.

Accordingly, the present inventors have provided for a partitioned imaginary sphere 150 at an optimal point that provides for optimal angling of insertion member 130 while maintaining point of contact 152 between head portion 132 and inner wall 62. This lessens, and optimally curbs, migration of wear debris. The inventors have also partitioned the imaginary sphere 150 at an optimal point that allows such angling of insertion member 130 while head 132 remains within opening 60 without protruding past first surface 54.

Conceptually, it is preferable to consider head 132 as a “slice” of a sphere 150 (as shown). It is also understood that the “slice” could come from an object that is toroidally-shaped, elliptically-shaped, globally-shaped (i.e., such as a non-perfect sphere), or otherwise be three-dimensional circular shape that has a slight curve or rounded portion at its outer edge . In the particular sphere-shaped embodiment shown in FIG. 10, if an imaginary sphere 150 was drawn around spherical head 132, the outer rim 138 of head 132 would fall directly on the sphere 150. Sphere 150 has a center point 154, and the “slice” that comprises spherical head 132 preferably contains center point 154 of sphere 150.

As shown in FIG. 9, it is even more preferable that center point 154 be slightly above the exact horizontal center of head 132. Without wishing to be bound to any theory, the present inventors believe that if the dimensions of the spherical or near-spherical head have already been provided, the appropriate corresponding taper section dimensions can be determined as described below. Alternatively, if the dimensions of a taper section of an opening have already been provided, the appropriate corresponding spherical or near-spherical head can be determined.

First, an imaginary line that represents a radius 156 of sphere is drawn from the center point 154 of imaginary sphere 150 superimposed on spherical or near-spherical head 132 to the outer rim 138 of head 132. Preferably, center point 154 of sphere 150 is located slightly above horizontal center of head 132 to provide the greatest angle available. A line tangent to the point where the radius 156 meets the outer rim 138 represents the tapered angle of extended frustoconical taper section 64. By providing a head 132 having a “slice” of the sphere 150 (or toroid, ellipse, or globe) that contains center point 154 at the position shown in FIG. 9, the most versatile angling positions of insertion member 130 are achieved, while still maintaining contact between head portion 132 and inner wall 62. It is also of benefit that outer rim 138 have more surface area than the tapered screws of the prior art, again providing the ability for insertion member 130 to be inserted and secured at a greater angle, while still maintaining contact with inner wall 62.

More specifically, as shown in FIG. 11, insertion member 130 can be rotated a substantial degree, while point of contact 152 maintains contact with inner wall 62. Although point of contact appears lower at some portions of insertion member 130 and higher at others, it still creates a friction lock or seal.

Various specific embodiments of insertion members are also shown in FIGS. 5-9. As stated, insertion members may be any member that is to be inserted into or through an opening 60. For example, insertion member may be screw 90 of FIG. 6, peg 110 of FIG. 7, or opening cover 120 of FIG. 8. Each of the screw 90, peg 110, and opening cover 120 have the above-described proprietary head configuration 132. Screw 90 and peg 110 also have a portion to be received in opening 60.

The use of this “universal-type” head 132 on screws 90, pegs 110, and opening covers 120 allows any of these insertion members to lock rigidly within openings 60. Moreover, because the invention provides a “universal-type” opening 60 that can be used with non-locking screws 100 or the various insertion members 130 according to this invention, it allows the surgeon a greater range of flexibility than that currently provided. By using the same cup 50, the surgeon has the ability to insert a peg, a screw, a bone spike, or a hole cover through any of openings 60 without interchanging cups. Additionally, the locking configuration of head 132 with extended taper section 64 provides an enhanced, liquid-tight lock.

In use, insertion member is positioned in opening 60 and an impact force is applied. This causes head 132 to seat against frustoconical taper 64 to form a liquid-tight seal, thereby restricting or at least partially limiting migration of wear debris through opening 60. The implantation techniques for implanting various insertion members varies depending upon patient considerations and surgeon preferences, and accordingly, will not be described further. Once cup 50 has been properly seated, the surgeon may secure liner 52 in cup through the use of known procedures. If necessary, the surgeon may also implant a femoral prosthesis to cooperate with the cup and liner, again, through the use of known procedures.

The foregoing is provided for purposes of illustrating, explaining, and describing embodiments of this invention. Modifications and adaptations to these embodiments will be apparent to those skilled in the art and may be made without departing from the scope or spirit of this invention or the following claims.

What is claimed is:

1. An implantable prosthesis, comprising:
(a) a prosthetic component having first and second surfaces, the second surface adapted to be oriented toward bone in which component is to be implanted;
(b) at least one opening extending from the first surface to the second surface; the opening adapted to receive an insertion member and comprising (i) an extended frustoconical taper section extending from the first surface through a substantial portion of the opening and (ii) a section at the second surface having a smaller diameter than the portion of the taper at the first surface, the opening adapted to accommodate an insertion member at multiple orientations relative to the component; and
(c) an insertion member having a spherical or near-spherical head and adapted to be inserted into the opening such that the spherical or near-spherical head interfaces with the extended frustoconical taper section.

2. The implantable prosthesis of claim 1, wherein once the appropriate orientation of the insertion member is selected, the insertion member is adapted to be locked relative to the frustoconical taper section such that the head of the insertion member does not protrude beyond the first surface.

3. The implantable prosthesis of claim 1, wherein the section at the second surface is a flat edge, a chamfered edge, a beveled surface, a rounded surface, or a spherical surface.

4. The implantable prosthesis of claim 1, wherein the spherical or nearspherical head comprises an outer edge that is spherical, near-spherical, toroidal, elliptical, global, slightly curved, or rounded.

5. The implantable prosthesis of claim 1, wherein the insertion member comprises one or more of a bone screw, a bone peg, a bone spike, or an aperture cover.

6. The implantable prosthesis of claim 1, wherein the spherical or near-spherical head comprises a sliced portion of a sphere having a center point and wherein the head includes the center point.

7. The implantable prosthesis of claim 1, wherein the opening has an inner wall, the spherical or near-spherical head has an outer rim, and wherein the insertion member is adapted to be inserted into the opening at an angle while maintaining a constant point contact between the inner wall and the outer rim.

8. The implantable prosthesis of claim 1, wherein the prosthesis comprises a hip replacement system and wherein the first and second surfaces are surfaces of an acetabular cup.

9. The implantable prosthesis of claim 1, wherein the interface between the spherical or near-spherical head and the extended taper section comprises a liquid-tight seal.

10. The implantable prosthesis of claim 1, wherein the opening is a universal-type opening and wherein the insertion member comprises any one of bone screw, a bone peg, a bone spike, or an aperture cover, wherein any one of the bone screw, a bone peg, a bone spike, or an aperture cover comprises a universal-type spherical or near-spherical head that corresponds to the universal-type opening.

11. An implantable prosthesis, comprising:
(a) a prosthetic component having first and second surfaces, the second surface adapted to be oriented toward bone in which component is to be implanted;
(b) at least one opening extending from the first surface to the second surface; the opening adapted to receive an insertion member and comprising (i) an extended frustoconical taper section extending from the first surface through a substantial portion of the opening and (ii) a rounded section at the second surface having a smaller diameter than the portion of the taper at the first surface, the opening adapted to accommodate an insertion member at multiple orientations relative to the component.

12. The implantable prosthesis of claim 11, wherein the opening is adapted to accommodate a spherical or near-spherical head insertion member such that the head of the insertion member interfaces with the extended frustoconical taper section.

13. An acetabular implant for fixation to a patient, comprising:
(a) an acetabular cup having an inner surface, an outer surface, and at least one opening extending from the inner surface to the outer surface, the outer surface adapted to be oriented toward bone in which component is to be implanted;
(b) the at least one opening having an extended frustoconical tapered section beginning at the inner surface; and
(c) a member for insertion into the opening, the member comprising a head having a spherically or near-spherically shaped portion that is adapted to interface with the extended frustoconical tapered section to lock and retain the insertion member at a desired orientation relative to the opening.

14. The acetabular implant of claim 13, wherein once the appropriate orientation of the insertion member is selected, the insertion member is adapted to be locked relative to the frustoconical taper section such that the head of the insertion member does not protrude beyond the first surface.

15. The acetabular implant of claim 13, wherein the spherical or near-spherical head comprises an outer edge that is spherical, near-spherical, toroidal, elliptical, global, slightly curved, or rounded.

16. The acetabular implant of claim 13, wherein the opening has an inner wall, the spherical or near-spherical head has an outer rim, and wherein the insertion member is adapted to be inserted into the opening at an angle while maintaining a constant point contact between the inner wall and the outer rim.

17. The acetabular implant of claim 13, wherein the interface between the spherical or near-spherical head and the extended taper section comprises a liquid-tight seal.

18. The acetabular implant of claim 13, wherein the member comprises a bone screw, a bone peg, a bone spike, or an opening cover.

19. An insertion member for use with a prosthesis having a universal connecting portion, comprising:
(a) a head having an outer edge shaped to approximate at least a portion of a sphere, a near-sphere, a toroid, an ellipse, a globe, a slight curve, or rounded portion; and
(b) a portion extending from the head adapted to be received by an opening of the prosthesis.

20. The insertion member of claim 19, wherein the insertion member comprises a bone peg, a bone screw, a bone spike, or an opening cover.

21. The insertion member of claim 19, further comprising a prosthesis having an opening with an extended frustoconical taper section and wherein the head of the insertion member is adapted to interface and lock with the opening.

22. The insertion member of claim 21, wherein the interface between the head and the extended taper section comprises a liquid-tight seal.

23. The insertion member of claim 19, wherein the head comprises a sliced portion of a sphere or a near-sphere having a center point and wherein the head includes the center point.

24. The insertion member of claim 19, wherein the opening has an inner wall, the head has an outer rim, and wherein the insertion member is adapted to be inserted into the opening at an angle while maintaining a constant point contact between the inner wall and the outer rim.

25. An acetabular implant for fixation to a patient, comprising:
(a) an acetabular cup having an inner surface, an outer surface, and at least one opening extending from the inner surface to the outer surface,
(b) the at least one opening having an extended frustoconical tapered section beginning at the inner surface and a second section at the outer surface having a diameter smaller than the diameter at the inner surface;
(c) a member for insertion into the opening, the member comprising a head having a spherically or near-spherically-shaped portion that is adapted to interface with the frustoconical extended tapered section to lock and retain the insertion member at a desired orientation relative to the opening.

26. The acetabular cup of claim 25, further comprising:
(d) a liner for lining the inner surface of the acetabular cup; and
(e) a femoral component for insertion into a patient's femur and adapted to cooperate with the acetabular cup and liner.

27. A method of replacing at least part of a hip joint in a patient, comprising:
(a) providing an acetabular cup having an inner surface, an outer surface, and at least one opening extending from the inner surface to the outer surface; the opening adapted to receive an insertion member and comprising (i) an extended frustoconical taper section extending from the first surface through a substantial portion of the opening and (ii) a section at the second surface having a smaller diameter than the portion of the taper at the first surface, the opening adapted to accommodate an insertion member at multiple orientations relative to the component; and
(b) providing at least one insertion member having a spherical or near spherical head and adapted to be inserted into the opening such that the spherical or near-spherical head interfaces with the extended frustoconical taper section;
(c) preparing the bone of the patient's hip to receive the acetabular cup; and
(d) implanting the acetabular cup, wherein once the appropriate orientation of the insertion member is selected, the insertion member is adapted to be locked relative to the frustoconical taper section.

28. The method of claim 27, wherein the head of the insertion member does not protrude beyond the first surface.

29. The method of claim 27, further comprising providing an acetabular cup liner and positioning the acetabular cup liner in the acetabular cup.

30. The method of claim 27, further comprising providing a femoral prosthesis and positioning the femoral prosthesis in a patient's femur, such that the femoral prosthesis can cooperate with the acetabular cup.

31. The method of claim 27, wherein once the appropriate orientation of the insertion member is selected, the insertion member is adapted to be locked relative to the frustoconical taper section such that the head of the insertion member does not protrude beyond the first surface.

32. The method of claim 27, wherein the section at the second surface is a flat edge, a chamfered edge, a beveled surface, a rounded surface, or a spherical surface.

33. The method of claim 27, wherein the spherical or near-spherical head comprises an outer edge that is spherical, near-spherical, toroidal, elliptical, global, slightly curved, or rounded.

34. The method of claim 27, wherein the insertion member comprises one or more of a bone screw, a bone peg, a bone spike, or an aperture cover.

35. The method of claim 27, wherein the spherical or near-spherical head comprises a sliced portion of a sphere having a center point and wherein the head includes the center point.

36. The method of claim 27, wherein the opening has an inner wall, the spherical or near-spherical head has an outer rim, and wherein the insertion member is adapted to be inserted into the opening at an angle while maintaining a constant point contact between the inner wall and the outer rim.

37. The method of claim 27, wherein the interface between the spherical or near-spherical head and the extended taper section comprises a liquid-tight seal.

38. The method of claim 27, wherein the opening is a universal-type opening and wherein the insertion member comprises any one of bone screw, a bone peg, a bone spike, or an aperture cover, wherein any one of the bone screw, a bone peg, a bone spike, or an aperture cover comprises a universal-type spherical or near-spherical head that corresponds to the universal-type opening.
